(19) United States
(12) Patent Application Publication
(10) Pub. No.: US 2020/0009043 A1
(43) Pub. Date: Jan. 9, 2020

(54) Cosmetic Composition for Keloid Scar Improvement

(71) Applicant: Young Oh Park, Seoul (KR)

(72) Inventor: Young Oh Park, Seoul (KR)

(21) Appl. No.: 16/491,596

(22) PCT Filed: Mar. 22, 2018
(86) PCT No.: PCT/KR2018/003343

§ 371 Date: Sep. 6, 2019

(30) Foreign Application Priority Data
Mar. 31, 2017 (KR) 10-2017-0041639

(51) Int. Cl. A61K 8/99; A61K 8/04; A61K 8/9794; A61Q 19/00

(52) U.S. Cl. CPC A61K 8/99; A61K 8/042; A61K 2800/805; A61Q 19/00; A61K 2800/82; A61K 8/9794

(57) ABSTRACT

The present invention relates to a cosmetic composition for keloid scar improvement. The cosmetic composition for keloid scar improvement according to the present invention contains a sponge cucumber extract and a pineapple extract as active ingredients. Through the above constitution, the cosmetic composition is prepared by using natural extracts, so that the present invention can have an excellent effect of improving keloid scars, cause fewer side effects, have antibacterial and anti-inflammatory actions of saponin and flavonoid components of sponge cucumber, relieve pain through the alleviation of skin itchiness and skin redness, and alleviate skin dryness through a moisturizing effect.

### TECHNOLOGICAL FIELD

The present invention pertains to a cosmetic composition to improve keloid scars, and more specifically the present invention pertains to a cosmetic composition to improve keloid scars, to ameliorate or improve scars by producing a cosmetic composition containing natural extracts by mixing sponge extracts and pineapple extracts.

### BACKGROUND TECHNOLOGY

Generally, if a certain level of wound occurs on the skin, occurrence of a scar cannot be avoided, and although severity of a scar depends on each individual's skin characteristics, the most important factor is severity of the initial wound.

Such formation of a scar is a normal biological reaction to a wound on the human skin, and such scar tissues could occur due to excessive growth of compact fibrous tissues in the general infection process. If a problem occurs in the process of leaving a normal scar, the scar could become excessively large, and such a scar could lower quality of life as well as cause inconvenient symptoms as the scar grows.

On the other hand, keloid is a disorder in which fibrous tissues grow abnormally densely in the wound healing process after damages to the skin occur and have the characteristic of growing beyond the size of the wound or area of the infection, and keloid could occur when a disorder occurs to the function that appropriately adjusts and restrains the wound healing process.

Keloid occurs mainly in black people and people who have dark skin color, and in those people with a genetic disposition (about 15% of the overall population) after they receive skin damages. Keloid not only causes cosmetic problems but could also obstruct the movement of joints in the event that it occurs in important areas such as the face or joints due to the fact that the area affected is broad.

Keloid or (keloid scar) occurs clinically as a hard knot of the skin color, as a hard hypochromic knot, or as a hard lupus knot, and unlike hypertrophic scars, keloid could invade the normal skin areas around the original scar area.

In terms of histopathologic diagnosis, with keloid (or keloid scar) the epidermis is normal, the thick skin is thick with sufficient vessels, compared to general scar tissue invasion of infected cells could increase, and whereas the collagen wads of the normal thick skin is loose with disorganized arrangement, keloid's thick skin wads are thick and abundant.

The most characteristic histologic explanation of keloid is that keloid is large collagen fibers arranged broadly and densely composed of countless fibrils, and thick hyalinized collagen that is irregularly arranged in the shape of a cyclone is called keloid collagen.

Proteoglycan, which is an important extra-cellular matrix other than collagen, is excessively embedded, and generally important symptoms to histopathologically diagnose keloid include the existence of hyalinized collagen, invasion of tongue-shaped collagen wads that encroach normal-looking epidermis and papillary dermis, horizontal cellular fiber bands visible on the upper reticular dermis, substantial fascial-shaped bands, and so on.

A hypertrophic scar occurs when the wound is healed as collagen is excessively accumulated in the affected areas to become larger than the normal size. A hypertrophic scar is dark in color, protrudes, and can turn into a soft scar. A hypertrophic scar occurs rapidly after a wound is sustained and could get better as time progresses. Also, a hypertrophic scar occurs only around the wound, occurs frequently, and is not related to the skin color.

Keloid treatments generally include injection of a steroid around the lesion, anticancer treatment, radioactive treatment, cryotherapy, surgeries, gradual amelioration of symptoms using surgery, silicon sheets, or ointments, and so on, but perfect treatment is considered to be impossible.

Especially, keloid scars must be approached differently from general scars. Although many patients selectively use scar ointments, it is difficult to use ointments easily due to the fact that onion extracts, which are the main ingredients of ointments, cause allergic reactions. Also, cosmetic products designed to ameliorate keloid using daily cosmetic products have not been developed in Korea yet. Furthermore, due to the fact that imported keloid cosmetic products are onion-based chemical products and cause allergic reactions, it is difficult to use these products daily, and they are expensive. Therefore, cosmetic products based on pure and natural ingredients that can be used daily are necessary.

On the other hand, a luffa cylindrical (or sponge) is a climbing plant belonging to the cucurbitaceae group and is known to contain various saponins, flavonoids, anthocyanins, and carotenoids as well as vitamin C, vitamin K, and many minerals. The above saponin component has excellent anti-inflammatory properties, and is effective in treating various skin ailments such as acne, eczema, atopy, and so on, and is especially effective in inhibiting skin infection affected by acne that has progressed to keloid.

Also, flavonoid, anthocyanin, and carotenoid have been reported to be effective in treating infections and acidifications. These components are produced by plants in order to protect themselves from external forces, and when these ingredients are applied to the human skin, they protect the skin from external stimulations by providing antibacterial, anti-inflammatory, and anti-acidic effects to improve the skin's immune system. Especially, these ingredients are known to be effective in ameliorating allergic reactions, colds, nasal cavity infections, pain, and various female disorders.

Also, pineapple is effective in treating digestion disorders and infections, and pineapple is one of the many traditional medicines that have been used widely for many centuries in the Central American and South American regions to treat these ailments. Pineapple's fruit and stem are rich in a protein-decomposing enzyme called bromelain. Bromelain mentioned above is especially abundantly contained in pineapple's stem and fruit, is a protein-decomposing enzyme, and is known to not only facilitate digestion but also to soften the skin and ameliorate swelling.

Thus, the inventor of the present invention produced a cosmetic composition that can be used conveniently everyday by using sponge extracts and pineapple extracts and completed the present invention by confirming that the present invention ameliorates and improves keloid scars.

Preceding technical documents of the present invention include Korean Registered Patent 10-1112051, Korean Registered Patent 10-1505294, Korean Publicized Patent 10-2015-0138721, and so on.

### DETAILED DESCRIPTION OF THE INVENTION

### Technical Issues

The purpose of the present invention is to provide a cosmetic composition to ameliorate or improve keloid scars by producing a cosmetic composition containing natural extracts produced by mixing sponge extracts and pineapple extracts.

Also, another purpose of the present invention is to provide a cosmetic composition to improve keloid scars that is excellent in improving keloid scars, that has little side effects, that has the antibiotic and anti-inflammatory effects of saponin and flavonoid components of sponge, that ameliorates pain by improving skin itchiness, reddening, and so on, and that improves skin dryness by protecting moisture.

Also, another purpose of the present invention is to provide a cosmetic composition to improve keloid scars that provides help in improving keloid scars by facilitating the softening of hardened skin with protein decomposing enzymes that are abundantly contained in pineapple extracts, and that can improve scars with steady daily use of the cosmetic composition rather than immediately treating scars.

The various issues that that present invention attempts to resolve are not limited to those issues mentioned above, and other issues that were not mentioned above can be clearly understood by those in the field in which the present invention belongs from the following information.

### Measures to Resolve the Issues

The cosmetic composition to improve keloid scars in accordance with the present invention contains as efficacious components sponge extracts and pineapple extracts.

The above sponge extracts could be produced using sponge skin, fibers, stems, eaves, and roots, and the above pineapple extracts could be produced by using pineapple skin, stems, fruit, and roots.

The above cosmetic composition could be used in cream products, gel products, cosmetic solution products, packs, or essence products.

For the above sponge extracts, a sponge is cleaned in order to remove foreign materials, and then the sponge is dried. Then, the above dried sponge is cut in a certain length and then pulverized, and the above pulverized sponge is hot water-extracted to produce sponge hot water extracts, and the above sponge hot water extracts are filtered using the low-temperature pressure reduction filtration method in order to eliminate the miniscule particles contained in the above sponge hot water extracts to produce sponge extracts, wherein with the above low-temperature pressure reduction filtration method the above sponge hot water extracts are cooled at the temperature of from 15 to 25° C. and then are filtered using a filter hole size of 0.1 to 1.0 μm under the pressure level of 0.1 to 0.5 kgf/cm², and then are refrigerated in the temperature of from −25 to −35° C. and dried in the powder state to produce sponge extracts. The above dried sponge extracts are dissolved in purified water, and then are filtered with a sterilization filter with the hole size of the filter of 0.2 μm to produce liquid sponge extracts.

The above liquid sponge extracts could be contained in the cosmetic composition in the amount of 0.01 to 90 weight %, and the above pineapple extracts could be contained in the cosmetic composition in the amount of 0.01 to 60 weight %.

The above sponge hot water extracts could be produced by putting the above pulverized sponge and purified water in the amount of 15 times to 20 times (W/V) of the weight of the above pulverized sponge into an extractor, by closing the above extractor, by keeping the temperature of the above extractor at 100 to 120° C. and at the temperature level of 1.2 to 1.5 kg/cm², and then by adding ultrasound waves with the vibration frequency of 30 to 50 KHz, and then by carrying out cyclical hot water extraction using an output of 100 to 200 watts for three to five hours.

The specific details of other embodiments are included in the detailed description.

### Effects of the Invention

The cosmetic composition to improve keloid scars in accordance with the present invention can ameliorate or improve keloid scars by producing a cosmetic composition containing natural extracts produced by mixing sponge extracts and pineapple extracts.

Also, the cosmetic composition to improve keloid scars in accordance with the present invention is produced by using natural extracts, so the composition is excellent in improving keloid scars, has little side effects, can ameliorate pain by improving such symptoms as skin itchiness and reddening, and improves skin dryness by protecting moisture.

Also, the cosmetic composition to improve keloid scars in accordance with the present invention facilitates softening of hardened skin by protein decomposing enzymes that are abundantly contained in pineapple extracts to provide help in improving keloid scars, and it is possible to improve scars by using the cosmetic composition daily rather than by using the cosmetic composition for immediate treatment purposes.

It can be sufficiently understood that various effects that are not specifically mentioned could be provided by the embodiments of the technological ideas of the present invention.

### BRIEF DESCRIPTION OF THE DRAWINGS

FIG. 1 is a graph that shows the average score of the efficacy evaluation in accordance with the comparative examples 1 through 3 and in accordance with the embodiments.

FIG. 2 is a photograph that shows the effects on the keloids around the chest of a 26-year-old male test subject in accordance with an embodiment of the present invention.

FIG. 3 is a photograph that shows the before-and-after test effects on the keloids around the chin of a 27-year-old male test subject in accordance with an embodiment of the present invention.

FIG. 4 is a photograph that illustrates the before-and-after test effects on the keloids around the chest of a 30-year-old male test subject in accordance with an embodiment of the present invention.

FIG. 5 is a photograph that shows the before-and-after test effects of on the keloids around the abdomen of a 54-year-old female test subject in accordance with an embodiment of the present invention.

FIG. 6 is a photograph that shows the before-and-after test effects on the keloids around the knee of a 34-year-old test subject in accordance with an embodiment of the present invention.

### EMBODIMENTS OF THE PRESENT INVENTION

The advantages and characteristics of the present invention, as well as the methods to achieve these will become clear when referring to the embodiments to be described in detail later. However, the present invention is not limited to the embodiments described herein, but could be embodied in other forms. Rather, the embodiments introduced here are provided in order to ensure that the information presented herein can become more rigorous and complete, and in order to ensure that the ideas of the present invention will be sufficiently communicated to those in the industry in which the present invention belongs.

The terminologies used in this application were used only in order to explain the specific embodiments, and the intention is not to limit the present invention. Singular terms include plural terms unless clearly indicated by the context.

Unless defined otherwise, all terminologies used herein including technical and scientific terminologies have the same meanings as those generally understood by those with an ordinary level of knowledge in the technological fiend in which the present invention belongs. Those terminologies that are defined by generally used dictionaries must be interpreted to have the same meanings as the meanings of the related technologies based on context, and otherwise clearly defined in this application, these terminologies are not interpreted to have ideal or excessively formal meanings.

Below, the keloid scar improving cosmetic composition in accordance with the present invention will be described in detail by referring to the attached drawings.

The keloid scar improving cosmetic composition in accordance with the present invention can be produced by including sponge extracts and pineapple extracts.

Under the present invention, the above sponge extracts could be dried by first removing foreign materials by washing a sponge.

Under the present invention, it is possible to use sponge skin, fibers, stems, leaves, and roots to obtain the above sponge extracts, and various sponge parts such as fruit and sap could be used as well.

Under the present invention, purified water with sodium bicarbonate (NaHCO₃) dissolved could be used for cleaning the above sponge, and the purified water in which the above sodium bicarbonate (NaHCO₃) is dissolved has the density range of 1 to 3 (w/w) %, and can be cleaned by being immersed for one minute to five minutes in the temperature of 25 to 35° C. Under the present invention, by washing the above sponge using the above sodium bicarbonate, it is possible to remove foreign materials attached to the above sponge, at the same time disinfecting fungi by expanding their cell walls.

It is possible to dry the above sponge by drying the sponge for three to eight hours in a dryer in which the temperature of 20 to 30° C. and the humidity of 30 to 35% are maintained in order for the moisture content of the above sponge to be reduced by 3 to 8 weight % compared to the initial entire weight of the sponge.

Next, it is possible to cut the above dried sponge in a certain length and pulverize.

Pulverization of the above dried sponge can be carried out by using any popular dry pulverizer, and it is possible to produce fragments by cutting the sponge in lengths of 5 to 10 mm. If pulverization of the above sponge is carried out in lengths of less than 5 mm, when carrying out the extraction process of the sponge the amount of miniscule particles increases to deteriorate workability, and if the length exceeds 10 mm, the extraction time lengthens.

Next, it is possible to produce sponge hot water extracts by hot water extracting the above pulverized sponge.

The above sponge hot water extracts could be produced by putting the above pulverized sponge and then the purified water of 15 to 25 times (W/V) the weight of the above pulverized sponge into an extractor, by closing the above extractor, by keeping the above extractor at the temperature of 100 to 120° C. and at the pressure level of 1.2 to 1.5 kgf/cm², by adding ultrasonic waves with the vibration frequency of 30 to 50 KHz, and by carrying out cyclical hot water extraction using 100 to 200 watts for three to five hours.

Under the present invention, in the event that the hot water extraction of the above sponge is carried out below the range mentioned above, it is possible that the useful components contained in the above sponge will not be effectively extracted, and if extraction is carried out beyond the above maximum range, increase in effect is insignificant, and the amount of miniscule particles increases when the above sponge extraction process is carried out to deteriorate workability.

Next, it is possible to remove small particles such as sponge extraction powders contained in the above sponge hot water extracts by filtering the above hot water extracts.

The above sponge hot water extract filtering can remove small particles contained in the sponge hot water extracts using the low-temperature pressure reduction filtration method. With the above low-temperature pressure reduction filtration method it is possible to produce sponge extracts by cooling the above sponge hot water extracts in the temperature of 15 to 25° C., by filtering using a filter with the filter hole size of 0.1 to 1.0 μm under the pressure level of 0.1 to 0.5 kgf/cm², and by freezing and drying the extracts.

Under the present invention, if the hole size of the above filter is less than 0.1 μm, the filter is ultra-precise and is high-cost, so is economically not feasible, and if the hole size exceeds 1.0 μm there is a lack of germ control, so fungi and bacteria could be included in the extracts, potentially causing pollution during storage or distribution. Also, with the above freezing and drying method, it is possible to produce sponge extracts by freezing and drying in the powder state the above filtered sponge hot water extracts in the temperature of −25 to −30° C.

Next, it is possible to produce liquid sponge extracts by dissolving in purified water the above frozen and dried sponge extracts and then filtering the extracts in a sterilized filter with the hole size of 0.2 um in order to produce the cosmetic composition.

Under the present invention, the above pineapple extracts could be produced by using the same method as the method used for producing sponge extracts, and it is possible to dry the pineapple after removing foreign materials by cleaning the pineapple.

Under the present invention, the above pineapple extracts could be produced by using pineapple skin, stems, fruit, and roots, and besides these other various pineapple parts could be used.

Under the present invention, the above cleaning of the pineapple could be carried out by using purified water in which sodium bicarbonate (NaHCO₃) is dissolved, and the above purified water in which sodium bicarbonate (NaHCO₃) is dissolved has the density range of 1 to 3 (w/w) %, and can be cleaned by being immersed from one minute to five minutes in the temperature of 25 to 35° C. Under the present invention it is possible to remove the foreign materials attached to the above pineapple, at the same time disinfecting fungi by expanding the cell walls by cleaning the above pineapple with the above sodium bicarbonate.

By drying the above pineapple for three to eight hours in a dryer that is kept at the temperature of 20 to 30° C. and humidity of 30 to 35%, the moisture content of the above pineapple is reduced by three to eight weight % relative to the initial weight of the pineapple.

Next, it is possible to cut the above dried pineapple in a certain length and pulverize it.

Pulverization of the above dried pineapple is carried out with any popular dry pulverizer, and it is possible to produce cut-pieces by cutting the pineapple in lengths of 5 mm to 10 mm. If pulverization of the above pineapple is carried out in lengths of less than 5 mm, during the pineapple extraction process the amount of small particles increases, thereby deteriorating workability, and if pulverization is carried out with lengths of less than 10 mm, the extraction time lengthens.

Next, it is possible to produce pineapple hot water extracts by hot water-extracting the above pulverized pineapple.

The above pineapple hot water extracts could be produced by putting the above pulverized pineapple and then the purified water of 15 to 25 times (W/V) the weight of the above pulverized pineapple into an extractor, by closing the above extractor, by keeping the above extractor at the temperature of 100 to 120° C. and at the pressure level of 1.2 to 1.5 kgf/cm², by adding ultrasonic waves with the vibration frequency of 30 to 50 KHz, and by carrying out cyclical hot water extraction using 100 to 200 watts for three to five hours.

Under the present invention, if the hot water extraction of the above pineapple is carried out in the range that is below the minimum range mentioned above, it will not be possible to effectively extract useful components contained in the above sponge, and if the hot water extraction is carried out in excess of the maximum range mentioned above, increase in effect is insignificant and the amount of small particles increases during the extraction process of the above pineapple, thereby deteriorating workability.

Next, it will be possible to eliminate small particles such as pineapple extract powders contained in the above pineapple hot water extracts by filtering the above pineapple hot water extracts.

It is possible to remove small particles contained in the pineapple hot water extracts by using the low-temperature pressure reduction filtration method. Under the above low temperature low pressure filtration method, it is possible to produce pineapple extracts by cooling the above pineapple hot water extracts in the temperature of from 15 to 25° C., by filtering using a filter with the filter hole size of 0.1 to 1.0 μm under the pressure level of 0.1 to 0.5 kgf/cm², and by freezing and drying the pineapple hot water extracts.

Under the present invention, if the hole size of the above filter is less than 0.1 μm, the filter is ultra-precise and is high-cost, so is economically not feasible, and if the hole size exceeds 1.0 μm there is a lack of germ control, so fungi and bacteria could be included in the extracts, potentially causing pollution during storage or distribution. Also, with the above freezing and drying method, it is possible to produce pineapple extracts by freezing and drying in the powder state the above filtered pineapple hot water extracts in the temperature of −25 to −30° C.

Next, in order to produce the cosmetic composition, the above frozen and dried pineapple extracts can be dissolved in purified water, and then can be filtered in a sterilized filter with the filter hole size of 0.2 μm to produce liquid pineapple extracts.

Below, embodiments of the keloid scar improvement cosmetic composition in accordance with the present invention will be described in more detail.

1. Production of Sponge Extracts and Pineapple Extracts

(1) Production of Liquid Sponge Extracts

Under the present invention, it is possible to dry the above sponge extracts by first cleaning the sponge to remove foreign materials.

First, after preparing sponge skin, fibers, stems, leaves, and roots, the sponge was cleaned by immersing the sponge for three minutes in the temperature of 30° C. using purified water with the density of 2 (w/w) % in which sodium bicarbonate (NaHCO₃) is dissolved.

Next, the solution was dried in a dryer in which the temperature of 25° C. and humidity of 33% are maintained for five hours, and using a dry pulverizer the sponge was cut in lengths of 5 mm to 10 mm to produce sponge pieces.

Next, after putting the above pulverized sponge pieces, and purified water with 20 times (W/V) the weight of the above pulverized sponge pieces in an extractor, the above extractor was closed, the above extractor's inside temperature and pressure were maintained at 110° C. and 1.3 kgf/cm², and after adding ultrasound waves with the vibration frequency of 40 KHz, cyclical hot water extraction was carried out for four hours using the output of 150 watts to produce sponge hot water extracts.

Afterwards, the above sponge hot water extracts were cooled at the temperature of 20° C. and were then filtered using a filter with the filter hole size of 0.2 μm under the pressure level of 0.3 kgf/cm², and then were frozen at the temperature of −28° C. to dry in the powder state in order to produce frozen and dried sponge extracts.

Next, in order to produce the cosmetic composition, the above frozen and dried sponge extracts were dissolved in purified water, and then the extracts were filtered in a sterilized filter with the filter hole size of 0.2 μm to produce the final liquid sponge extracts with 10 weight %.

(2) Production of Liquid Pineapple Extracts

Liquid pineapple extracts were produced by using the same method as the production method for the above liquid sponge extracts.

2. Production of the Cosmetic Composition Specimen Containing Sponge Extracts and Pineapple Extracts

As shown in [Table 1] below, the basic cosmetic solution composition was produced, and this was used for the embodiment, and for comparative example 1 to comparative example 3.

The basic cosmetic solution's components are 5 weight % of butylene glycol, 0.5 weight % of sodium hyaluronate, 5 weight % of glycerin, 3 weight % of 1,2-hexanediol and refined water for the remainder, and solutions of the active ingredients sponge extracts and pineapple extracts each at 10 weight % were produced.

Comparative example 1 is a control group that does not contain either the sponge extracts or the pineapple extracts, comparative example 2 contains 20 weight % of sponge extracts, comparative example 3 contains 20 weight % of pineapple extracts, and comparative example 4 contains 20 weight % of sponge extracts and 20 weight % of pineapple extracts.

TABLE 1
Cosmetic solution specimens' component ratios

| | | Contents (weight %) | | | |
|---|---|---|---|---|---|
| | | Compara- | Compara- | Compara- | |
| | | tive | tive | tive | Embodi- |
| Number | Components | example 1 | example 2 | example 3 | ment |
| 1 | Sponge | 0 | 20 | 0 | 20 |
| | extracts | | | | |
| 2 | Purified water | 86.5 | 66.5 | 66.5 | 46.5 |
| 3 | Pineapple | 0 | 0 | 20 | 20 |
| | extracts | | | | |
| 4 | Butylene glycol | 5 | 5 | 5 | 5 |
| 5 | Sodium | 0.5 | 0.5 | 0.5 | 0.5 |
| | hyaluronate | | | | |
| 6 | Glycerin | 5 | 5 | 5 | 5 |
| 7 | 1,2-hexanediol | 3 | 3 | 3 | 3 |
| Total | | 100 | 100 | 100 | 100 |

<Example of a Test> Clinical Trial Using the Cosmetic Composition Specimen Containing Sponge Extracts and Pineapple Extracts in Patients with Keloid

As shown in [Table 1] above, after producing the cosmetic compositions in comparative example 1 through 3 and in the embodiment, each cosmetic composition was provided to five test subjects for the total number of 20 test subjects to be used for one month in order to evaluate the efficacy of each composition.

In the above test examples, cosmetic compositions were produced in the form of mists to be applied to the affected areas, and the method of usage was to apply each composition by spraying evenly to the affected areas five times a day. For evaluation, “0” was given if there was no effect, and “10” was given if the effect was substantial based on the test subject's opinion.

There were three evaluation items, which were A. amelioration of reddening and brightening effect, B. amelioration of scars and pus acne, and C. softening of scars.

The evaluation results of comparative example 1 to comparative example 3 are indicated in [Table 2] to [Table 4], and the evaluation results of the embodiment are indicated in [Table 5].

TABLE 2
Evaluation of clinical effectiveness of comparative example 1

| | | Test subjects | | Efficacy evaluation | |
|---|---|---|---|---|---|
| Number | Specimen | Sex (age) | Keloid areas | Items* | Scores** |
| 1 | Comparative | Male (28) | Chin | A | 2 |
| | example 1 | | | B | 1 |
| | | | | C | 2 |
| 2 | | Male (37) | Chest | A | 0 |
| | | | | B | 1 |
| | | | | C | 3 |
| 3 | | Male (30) | Back | A | 2 |
| | | | | B | 2 |
| | | | | C | 2 |
| 4 | | Female (27) | Chest | A | 2 |
| | | | | B | 0 |
| | | | | C | 1 |
| 5 | | Female (43) | Chest | A | 3 |
| | | | | B | 2 |
| | | | | C | 2 |

*Items: A. Amelioration of reddening and brightening, B. Amelioration of scars and pus acne, C. Amelioration of scars
**Scores: 0. No effect, 10. Substantial effect

TABLE 3
Evaluation of clinical effectiveness of comparative example 2

| | | Test subjects | | Efficacy evaluation | |
|---|---|---|---|---|---|
| Number | Specimen | Sex (age) | Keloid areas | Items* | Scores** |
| 1 | Comparative | Male (25) | Chin | A | 7 |
| | example 1 | | | B | 5 |
| | | | | C | 4 |
| 2 | | Male (32) | Chin | A | 6 |
| | | | | B | 5 |
| | | | | C | 4 |
| 3 | | Male (32) | Chest | A | 6 |
| | | | | B | 6 |
| | | | | C | 4 |
| 4 | | Female (24) | Chest | A | 5 |
| | | | | B | 5 |
| | | | | C | 3 |
| 5 | | Female (33) | Chest | A | 7 |
| | | | | B | 6 |
| | | | | C | 4 |

TABLE 4
Evaluation of clinical effectiveness of comparative example 3

| | | Test subjects | | Efficacy evaluation | |
|---|---|---|---|---|---|
| Number | Specimen | Sex (age) | Keloid areas | Items* | Scores** |
| 1 | Comparative | Male (27) | Chin | A | 2 |
| | example 3 | | | B | 2 |
| | | | | C | 6 |
| 2 | | Male (30) | Chin | A | 3 |
| | | | | B | 3 |
| | | | | C | 5 |
| 3 | | Male (32) | Chest | A | 3 |
| | | | | B | 3 |
| | | | | C | 6 |
| 4 | | Female (28) | Chest | A | 4 |
| | | | | B | 3 |
| | | | | C | 7 |
| 5 | | Female (38) | Back | A | 2 |
| | | | | B | 3 |
| | | | | C | 6 |

TABLE 5
Clinical effectiveness of the embodiment

| | | Test subjects | | Efficacy evaluation | |
|---|---|---|---|---|---|
| Number | Specimen | Sex (age) | Keloid areas | Items* | Scores** |
| 1 | Embodiment | Male (26) | Chest | A | 8 |
| | | | | B | 7 |
| | | | | C | 7 |
| 2 | | Male (27) | Chin | A | 8 |
| | | | | B | 8 |
| | | | | C | 7 |
| 3 | | Male (30) | Chest | A | 8 |
| | | | | B | 6 |
| | | | | C | 7 |
| 4 | | Female (54) | Abdomen | A | 9 |
| | | | | B | 7 |
| | | | | C | 9 |
| 5 | | Female (34) | Knee | A | 10 |
| | | | | B | 8 |
| | | | | C | 5 |

FIG. 1 is a graph that shows the average scores of effectiveness evaluations of comparative example 1 to 3 and of the embodiment.

According to the above [Table 2] to [Table 5] and FIG. 1, based on the evaluation results it was not possible to confirm the overall effectiveness since all three items were 0 to 3 or less in comparative example 1 [Table 2] for which only the basic cosmetic composition was used, and in comparative example 2 [Table 3] only sponge extracts were added to the basic cosmetic solution, and for evaluation item A the average score was 6.2, for B the score was 5.4, and for C the score was 3.8, so it was confirmed that there were some effects regarding the amelioration of reddening and amelioration of scars, but it was not possible to confirm whether there were any effects on the softening of scars.

Comparative example 3 [Table 4] contains only pineapple extracts in the basic cosmetic solution, and for evaluation item A the average score was 2.8, for B the average score was 2.8, and for C the score was 6.0, so it was confirmed that the solution is effective only in softening scars. The embodiment [Table 5] contains the sponge extracts and pineapple extracts in the basic cosmetic solution, and for item A the average score was 8.6, for B the average score was 7.2, and for C the average score was 7.0, so we could confirm that the solution is effective in all three evaluation items.

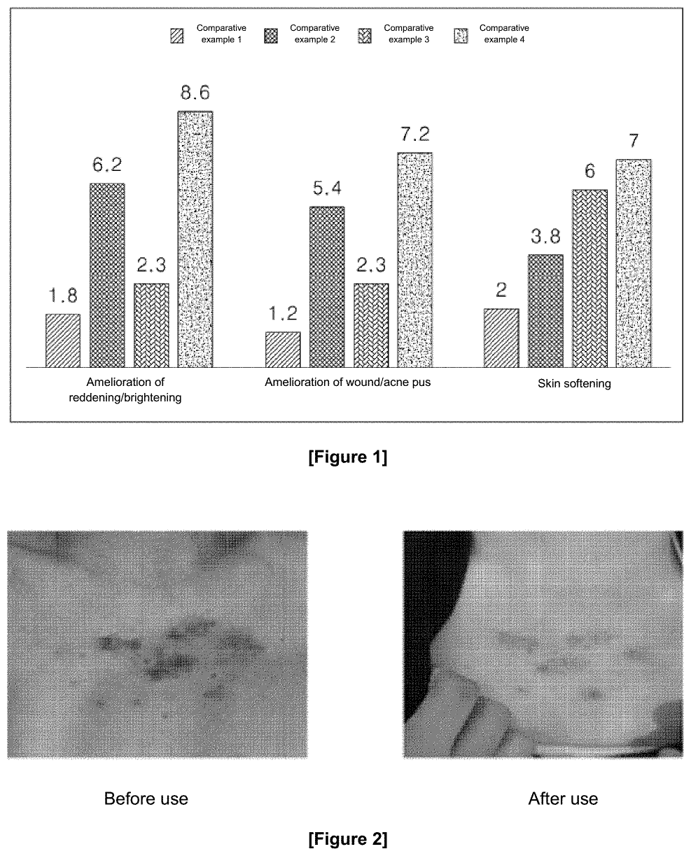

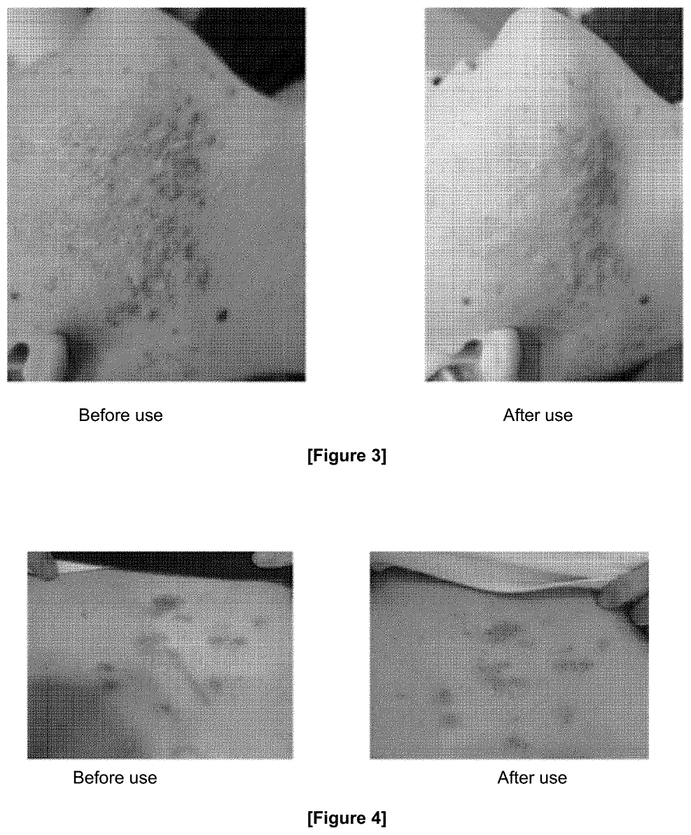

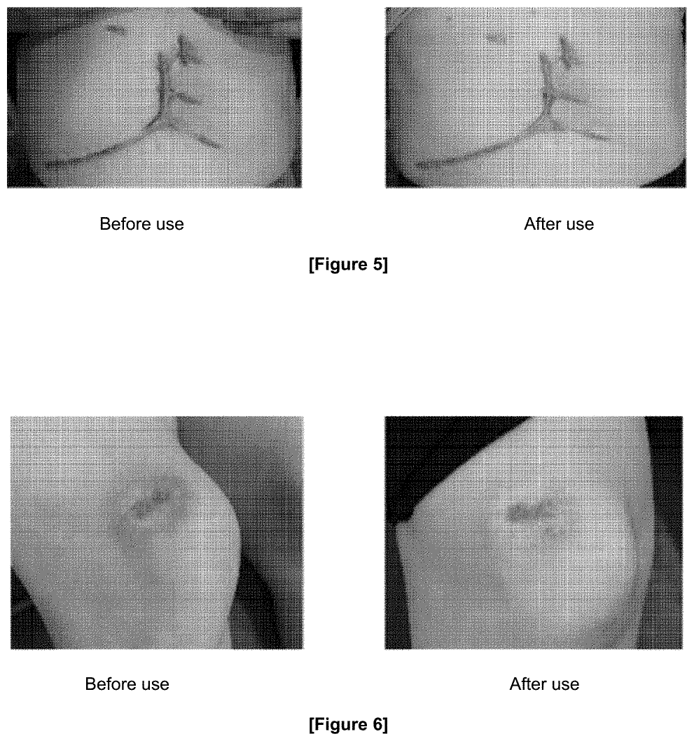

FIG. 2 through FIG. 6 show the before-and-after photographs of the cosmetic solution produced in accordance with the embodiment.

When we refer to FIG. 2 to FIG. 6, we can see that keloid scars were effectively improved in those test subjects that used the cosmetic solution produced by mixing sponge extracts and pineapple extracts in accordance with the embodiment of the present invention

### Production Example

Production of a Cosmetic Product Containing Sponge Extracts and Pineapple Extracts

As examples of the cosmetic solution using the natural extracts produced by mixing sponge extracts and pineapple extracts in accordance with the present invention, mist essences and ampules containing sponge extracts and pineapple extracts were produced.

The above mist essence was produced with the sponge extract solution containing 0.1 to 0.3 weight % of sponge extracts comprising 70% of the overall volume, and with the pineapple extract solution containing 0.1 to 0.3 weight % of pineapple extracts comprising 20% of the entire volume.

The above ampule was produced with the above sponge extract solution comprising 50% of the overall volume and with the above pineapple extract solution comprising 40% of the overall volume.

Although an advisable embodiment of the present invention was described above, those with an ordinary level of knowledge in the technological field in which the present invention belongs understand that it is possible to embody the present invention in other specific forms without changing the technological ideas or required characteristics. Therefore, it must be understood that the embodiment described above is illustrative in all aspects and does not limit the present invention.

1. A cosmetic solution to improve keloid scars containing sponge extracts and pineapple extracts.

2. A cosmetic solution to improve keloid scars according to claim 1, wherein the above sponge extracts are produced using sponge skin, fibers, stems, leaves, and roots, and wherein the above pineapple extracts are produced using pineapple skin, stems, fruit, and roots.

3. A cosmetic composition to improve keloid scars according to claim 2, wherein the above cosmetic solution is used in creams, gels, cosmetic solutions, packs, or essence products.

4. A cosmetic composition to improve keloid scars according to claim 3, wherein the above sponge extracts are produced by cleaning the sponge to remove foreign materials and drying, by cutting the above dried sponge in a certain length and pulverizing the sponge, by hot water-extracting the above pulverized sponge to produce sponge hot water extracts, by filtering the above sponge hot water-extracts using the low temperature and pressure reduction filtration method, and by removing the small particles contained in the above hot water extracts, wherein the above low temperature pressure reduction filtration method the above sponge hot water extracts are cooled at the temperature of 15 to 25° C. and filtered with the filter hole size of 0.1 to 1.0 μm under the pressure level of 0.1 to 0.5 kgf/cm² and afterwards are frozen at the temperature of −25 to −30° C. to dry in the powder state, and by dissolving the above frozen and dried sponge extracts in purified water, and then by filtering the sponge extracts in a sterilizer filter with the filter hole size of 0.2 μm to produce liquid sponge extracts.

5. A cosmetic solution to improve keloid scars according to claim 4, wherein the above liquid sponge extracts are contained in the cosmetic composition in the amount of 0.01 to 90 weight %, and wherein the above pineapple extracts are contained in the amount of 0.01 to 60 weight % in the cosmetic composition.

6. A cosmetic composition to improve keloid scars according to claim 5, wherein the above sponge hot water extracts are produced by putting the above pulverized sponge, and purified water with 15 times to 25 times (W/V) the weight of the above pulverized sponge, by closing the above extractor afterwards, by keeping the temperature and pressure of the inside of the above extractor at 100 to 120° C. and 1.2 to 1.5 kgf/cm², by adding ultrasound waves with the vibration frequency of 30 to 50 KHz, and by carrying out cyclical hot water extraction using 100 to 200 watts for three to five hours.
